(19) United States
(12) Patent Application Publication
(10) Pub. No.: US 2015/0335469 A1
(43) Pub. Date: Nov. 26, 2015

(54) THERMOREGULATION INTERFACE PACK AND ASSEMBLY

(71) Applicant: Physiolab Technologies Limited, Milton Keynes, Buckinghamshire (GB)

(72) Inventors: Nicholas Rose, Milton Keynes, Buckinghamshire (GB); Mark Haskins, Milton Keynes, Buckinghamshire (GB)

(21) Appl. No.: 14/410,268

(22) PCT Filed: Jun. 24, 2013
(86) PCT No.: PCT/GB2013/051659

§ 371 Date: Dec. 22, 2014

(30) Foreign Application Priority Data
Jun. 22, 2012 (GB) 1211149.8

(51) Int. Cl. A61F 7/02

(52) U.S. Cl. CPC A61F 7/02; A61F 2007/0054

(57) ABSTRACT

A thermoregulation interface pack (10) for the treatment of physical injuries and disorders includes a plurality of conduits (12-16) providing separate feed and return through channels (24, 26), are arranged in each conduit in a plurality of spiral shapes (30), each of which provides a fluid path inversion (32). At the fluid path inversion (32) the conduits (12-16) are provided with flow constrictors (50). This shape of the conduits (12-16) provides an array of zones (30) of zero fluid speed which optimises energy transfer between the interface pack (10) and the patient. The interface pack (10) is preferably formed of two layers (60, 62) of material of which the inner or contact layer (62) is made of a more conformable material than the overlying or upper layer (60), causing the contact layer (62) to deform to a greater extent than the upper layer (60), thereby to increase surface contact with the patient. The interface pack (10) can be provided with an insulating layer (70), with pressure release valves (88) and with a compression sleeve (100).

### TECHNICAL FIELD

The present invention relates to a thermoregulation interface pack and assembly for the treatment of physical injuries and disorders. The preferred embodiments are also able to effect pressure treatment on a patient.

### BACKGROUND ART

There are numerous instances where it is desired to effect a thermal treatment on a patient. For example, this may be to treat a physical injury, such as of the muscles, ligaments, tendons and the like. It may also be useful in treating skin injuries, as well as illnesses such as infections and the like.

Thermal treatments of this type have been known for many years, in their simplest form being ice packs. Subsequently, heat generating packs were developed, typically in the form of a pouch of chemical material which can be made to react exothermically and thereby to release heat. These devices are intended to cool or heat, as appropriate, a part of a person's body in order to alleviate inflammation, pain suffered by the patient and so on. It has been found that if properly applied, such treatments can significantly minimise patient discomfort caused by the injury or illness as well as speed up the recovery process. However, such ice packs and heat generating packs provide a relatively crude form of temperature regulation, unable to provide optimum treatment of an injury without constant and close monitoring by a medical practitioner.

More recently, attempts have been made to develop thermoregulation devices which have some form of built-in control, for example in which a desired treatment temperature can be set in a control unit and then used to feed energy to a cuff or pad. This may be by means of heated or cooled fluid or by electrical heating or cooling. An early example known to the applicant is DE-3,343,664. Other examples include EP-0,812,168, U.S. Pat. No. 6,818,012 and EP-2,393,459.

While such control systems are known and have attempted to provide accurately controllable temperature regulation at the patient's skin, there are numerous variables which result in such systems being inaccurate. Moreover, in such systems, particularly fluid based systems, it has been found difficult to produce the desired temperature at the actual site of the patient to be treated. This is caused by a number of factors, of which the primary include difficulties in controlling the flow of fluid in an applicator cuff, difficulties in ensuring proper energy transfer to the patient through the cuff, speed of energy transfer to the patient with consequential speed of adjustment of treatment parameters, and so on. If these difficulties could be overcome, it is believed that fluid based systems could provide the most effective form of thermoregulation.

There have been a number of attempts to design cuffs suited to such thermoregulation systems, including for instance EP-1,929,980 and US-2006/0191675. However, these designs to not generally resolve the problems indicated above.

### SUMMARY OF THE PRESENT INVENTION

The present invention seeks to provide an improved thermoregulation interface pack and assembly for the treatment of physical injuries and disorders. The preferred embodiments are also able to effect pressure treatment.

According to an aspect of the present invention, there is provided a thermoregulation interface pack provided with a treatment surface having a treatment zone; at least one fluid conduit in the interface pack and extending across the treatment zone; the fluid conduit including a plurality of path inversions disposed in the treatment zone, and flow constrictors disposed proximate at least some of the path inversions.

The inventors have found that some of the principal problems of fluid based thermoregulation interface pack include that such packs do not adequately control the flow, location or heat transfer to or from the fluid in the interface pack. For example, with interface packs having relatively large fluid chambers, it is not possible to control the location of fluid in the interface pack, particularly when it is pressed against a patient's body, nor the flow of fluid in the interface pack. This results in the generation of zones of the interface pack which do not provide adequate energy transfer to and from the patient. These underperforming zones are often at or proximate the point where the patient most requires the treatment. With interface packs which provide continuous fluid flow through the interface pack, for example through conduits, much of the available energy is wasted by being transferred through the moving fluid rather than being released for treatment. Moreover, such interface packs do not resolve the problem of pressure on the pack altering the flow of fluid to create ineffective zones in the interface pack.

By contrast, the structure taught herein provides, at the points of path inversion, zones in the fluid conduit of zero apparent fluid flow but without actually stopping the flow of fluid in the conduit. More specifically, fluid flowing in the conduit has to invert its flow direction due to the path inversions and will thus have a point of zero instantaneous speed. The flow restrictors create turbulence in the fluid at the points of path inversion, which ensure that the fluid continues to move rather than stagnate, avoiding the generation of laminar fluid flow and maximising the mixing of fluid at the points of path inversion, thereby optimising the energy transfer to or from the fluid through the walls of the interface pack.

The term cuff as used herein is intended also to encompass pads, sleeves and garments designed to be placed on or around a part of a patient's body, such as a limb or the like.

Advantageously, flow constrictors are provided at each path inversion in the treatment zone and they are preferably located at the point of inversion. This arrangement optimises the structure, although it is not excluded, for example, that the flow restrictors could be located upstream or downstream of the points of path inversion. The greater the distance of the flow restrictors from the point of path inversion, the lesser effect they will have at the point of inversion.

It is preferred that the points of path inversion are generally uniformly disposed in the treatment zone, most preferably in a regular array across the area of this zone.

The treatment zone may extend for the whole of the treatment surface of the interface pack, but may also constitute only a part of the treatment surface.

In the preferred embodiment, the or each conduit is in the form of a series of spirals with each spiral curving in opposing directions either side of a point of path inversion. This structure has been found to be the most effective in that it creates a series, in the preferred embodiment an array, of thermo-regulated zones in the interface pack. This shape of the conduit has been found to create very effective heat transfer zones in the pack, much better able to transfer heat to and from the fluid in the pack and thus to and from the patient. Moreover, it has been found that this shape can provide rapid changes in fluid temperatures in the pack, enabling it to be used in treatments which provide sophisticated and variable temperature profiling, not possible with prior art structures. This shape is also able to transmit treatment pressure to the patient, generated by the pressure of the fluid supply as described below.

Advantageously, the flow constrictors are in the form of a narrowing of the conduit, for instance in the form of pinching of the walls of the conduit. In other embodiments, the flow constrictors could be provided by one or more baffles within the conduit or other suitable elements.

Advantageously, the interface pack includes first and second layers forming the interface pack, the second layer providing the treatment surface and the first layer providing an outer layer of the interface pack, wherein the first layer has a stiffness greater than a stiffness of the second layer at least in the treatment zone. This feature ensures than pressure of fluid in the interface pack will cause the second layer, and hence the treatment surface, to deform in preference to the first surface, thereby providing enhanced contact of the interface pack against a patient's skin. Advantageously, the second layer is made of a conformable material. In the preferred embodiment, the second layer is thinner than the first layer, leading to its increased flexibility. Other embodiments have the first and second layers of different materials, which may or may not be of different thicknesses.

There may be provided an insulation layer disposed across the first layer, preferably over the outer layer thereof.

In an embodiment, the interface pack is provided with one or more pressure relief valves. Advantageously, a plurality of pressure relief valves is provided within the conduit, with one or more most preferably in the treatment zone. It is preferred that the or each pressure relief valve is in the form of an aperture in the first layer of the interface pack, with a pressure removable cover over the aperture. The cover may be an adhesive tab, which adhesive is chosen to release upon exceeding of a threshold pressure.

It is preferred that the pressure relief valves are covered by the insulation layer; which results in any loss of fluid from the interface pack as a result of opening of the pressure relief valves being held by the insulation layer. Advantageously, the insulation layer is fluid tight and is separate from the first layer at least in the locations of the pressure relief valves, so as to create chambers for holding pressure released fluid. This ensures that fluid does not leak out of the interface pack and thus that the interface pack can remain operational even after opening of one or more of the pressure relief valves.

In a preferred embodiment, there is provided a compression element for pressing the treatment surface against a patient. The compression element may be a pressure sleeve or belt. In the preferred embodiment, the compression element includes a plurality of compression belts arranged in a longitudinal sequence of the interface pack.

In an embodiment, there is provided a gel layer overlying the treatment surface and for contact with a patient. The gel layer promotes good thermal contact between the interface pack and the patient's skin.

Other features of the teachings herein will become apparent to the skilled person from the specific description which follows.

### BRIEF DESCRIPTION OF THE DRAWINGS

Embodiments of the present invention are described below, by way of example only, with reference to the accompanying drawings, in which:

FIG. 1 is a plan view of a preferred embodiment of interface pack;

FIG. 2 is a schematic view of an embodiment of conduit for an interface pack according to the present invention;

FIG. 3 is a plan view of the interface pack of FIG. 1 in further detail;

FIG. 4 shows various details of one example of the of FIG. 1;

FIGS. 5A and 5B show cross-sectional views of the structure of an embodiment of interface pack;

FIG. 6 is a cross-sectional view of another embodiment of interface pack provided with an insulation layer;

FIGS. 7A and 7B is a cross-sectional view of another embodiment of interface pack provided with an insulation layer and pressure relief valves;

FIG. 8 shows various cross-sectional views of the structure of another embodiment of interface pack;

FIG. 9 is a schematic view of an embodiment of pressure sleeve for the interface pack disclosed herein;

FIGS. 10 to 12 are views showing different details of the pressure sleeve of FIG. 9;

FIG. 13 is a schematic diagram showing the effect of the use of gel to enhance the thermal contact of the interface pack against a patient's skin; and

FIG. 14 is an exploded view of the various layers of an embodiment of interface pack.

### DESCRIPTION OF THE PREFERRED EMBODIMENTS

Described below are various embodiments of temperature regulated interface pack, which is designed to be conformed around a part of a patient's body such as a limb or the like. The teachings therein, however, are not limited to an interface pack of a specific form, as the interface pack could have any shape suitable for the particular treatment desired for a patient. In some embodiments, the interface pack could be in the form of a sleeve or garment into which part of the patient's body to be treated can be inserted.

The interface pack is intended for use with a temperature regulation system which includes a pump, heating and/or cooling elements for heating fluid which is then pumped via the pump into the conduits of the interface pack. It is envisaged that such a system would provide one or more temperature sensors able to sense the temperature of fluid in the interface pack or the temperature of the interface pack. Such sensors may be provided within the system or as part of the interface pack itself.

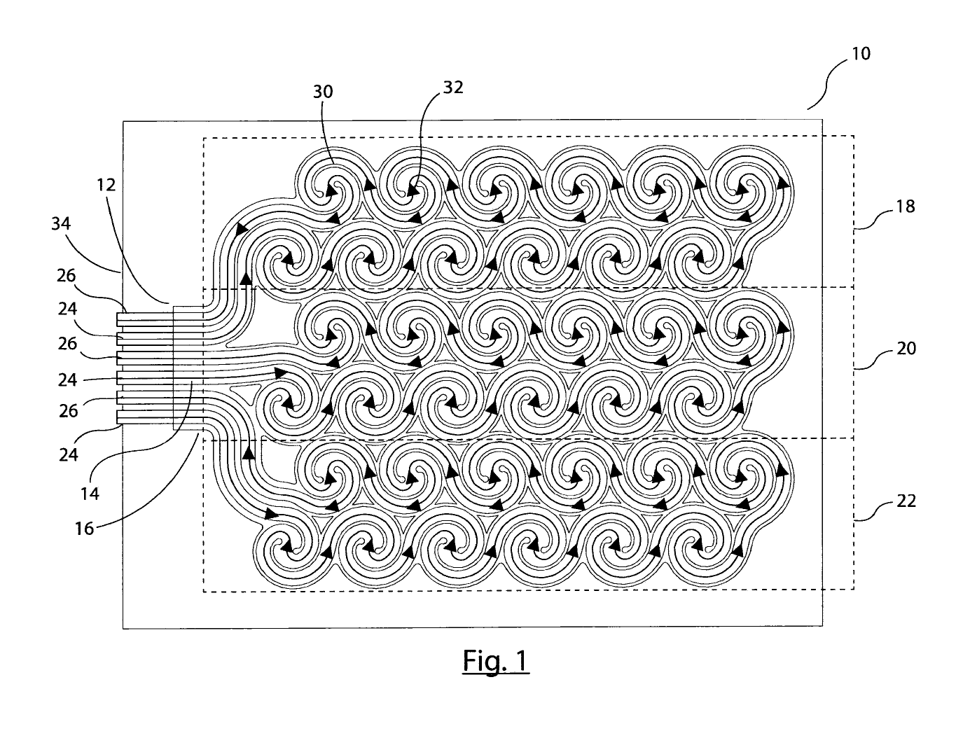

Referring to FIG. 1, this shows a preferred embodiment of interface pack 10 in schematic form. The interface pack may be a substantially planar structure, preferably flexible to be conformable to a patient, and may have other forms as well, such as a cuff or garment part. The interface pack 10 is formed of two layers of impermeable material, advantageously a polymer, which are bonded together to form therewithin a plurality of conduits 12, 14, 16, each of which extends in a respective temperature regulation treatment zone 18, 20, 22. Each conduit 12-16 includes a respective feed path 24 and return path 26. It will be appreciated that the number of conduits 12-16 and the number of treatment zones 18-22 may vary with, in some cases, there being only a single conduit and treatment zone, while in other cases there may be provided two or more than three sets of conduits and treatments zones. Moreover, the treatment zones 18-22 may not necessarily be aligned side-by-side as in the embodiment of FIG. 1, as these could be arranged in an array or any other manner desired or optimal for a particular application.

The conduits 12-16, and as a result the treatment zones 18-22, in this embodiment extend over substantially entire surface area of the interface pack 10. In other embodiments, the temperature regulation treatment zones 18-22 may extend only over a part of the interface pack 10, for instance but not necessarily in a central portion thereof.

With respect to the embodiment shown in FIG. 1, each conduit 12-16 is arranged in a plurality of spirals 30. In this example six spirals 30 are disposed in what could be termed the feed direction and six spirals disposed in what could be termed the return direction of the conduit, for a total of twelve spirals to each conduit 12-16. Again, the number of spirals 30 would be dependent upon the size of the interface pack 10 in particular of the heat treatment zones.

Each spiral 30 provides a forward and a return path therein and a path inversion at point 32, located at the centre-point of the spiral. Thus, in this example, each conduit or fluid path 12-16 provides twelve path inversions along its length. The advantages of these is described in detail below.

As will be apparent from FIG. 1, the conduits 12-16 are all aligned at a common coupling zone 34 of the interface pack 10, for coupling to a suitable fluid source. With this embodiment, thus, the fluid source would typically include a suitable manifold providing three fluid inlets and three fluid return paths. These may be commonly connected so as to provide equivalent fluid flows through the conduits 12-16 but in other embodiments may be separately fed so as to provide different fluid flows through each of the conduits and thus in each of the temperature regulation treatment zones 18-22.

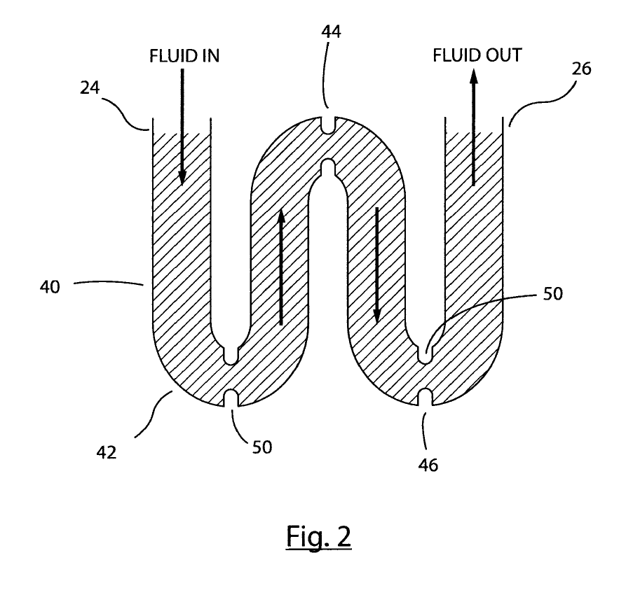

Referring now to FIG. 2, there is shown another embodiment of conduit 40 having similarities to the conduits 12-16. However, instead of having spiral paths 30, the conduit 40 has what could be described as a zigzag shape, with a plurality of path inversions 42-46. It will be appreciated that in practice the conduit of the example of FIG. 2 would have many more points of inversion 42-46 than shown in FIG. 2, which is to be taken as schematic only. The conduit 40 of FIG. 2 is less preferred than the arrangement of FIG. 1, as spiral paths of the type shown in FIG. 1 provide distinct regions within each of the conduit paths 12-16, which have been found to maximize the transfer of energy to and from fluid in the conduits 12-16 and thus to and from a patient. It has also been found that spiral paths provide good pressure control of the interface pack 10, as described in further detail below. Even if not preferred, the arrangement of FIG. 2 can be suitable for a number of applications.

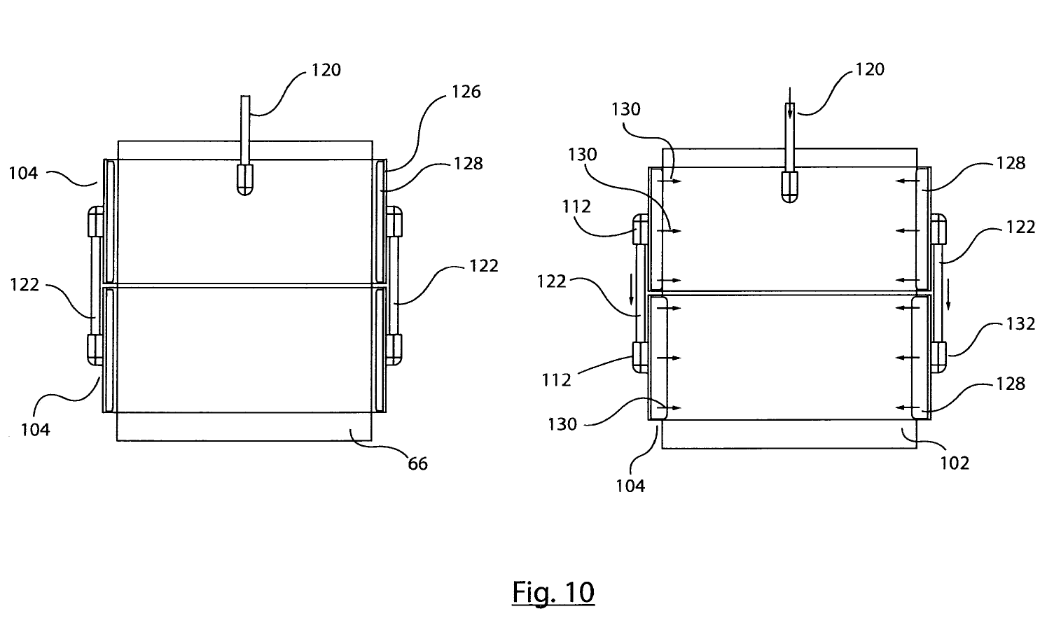

FIG. 2 also shows an example of flow constrictor 50, which may also be provided in the conduits 12-16 of the embodiment of FIG. 10. The flow constrictors are located at the points 32 of path inversion. As shown in FIG. 2, in a preferred embodiment, the flow constrictors 50 are what can be described as pinched zones of conduit wall, which provide a restriction to the path of fluid through the conduit. In other embodiments, the flow restrictors 50 could be baffles within the conduit. The purpose of the flow restrictors 50 is to generate turbulence at the point of path inversion. This turbulence ensures that there is no stagnation of fluid at the point of path inversion, which could otherwise lead to the generation of laminar flow, which would contribute to loss of thermal transfer efficiency of the interface pack.

In the embodiment of FIG. 2, as with the other embodiments of this invention, the flow constrictors 50 are preferably located substantially precisely at the points of path inversion. The flow constrictors could, on the other hand, be disposed just upstream or downstream of the points of path inversion and in this regard any location of the flow restrictors 50 which creates turbulence at the points of path inversion would be suitable. It is considered, though, that optimum turbulence is created with the constrictors precisely at the points of path inversion.

The narrowing of the conduit 40 produced by the flow constrictors 50 could be to one side only of the conduit 40, but preferably both sides are constricted as shown in the drawings and clearly visible in FIG. 2. It is not considered necessary to provide flow constrictors at the top and bottom walls of the conduit 40 but this is not an excluded possibility. It will be appreciated that these features are applicable to all embodiments taught herein.

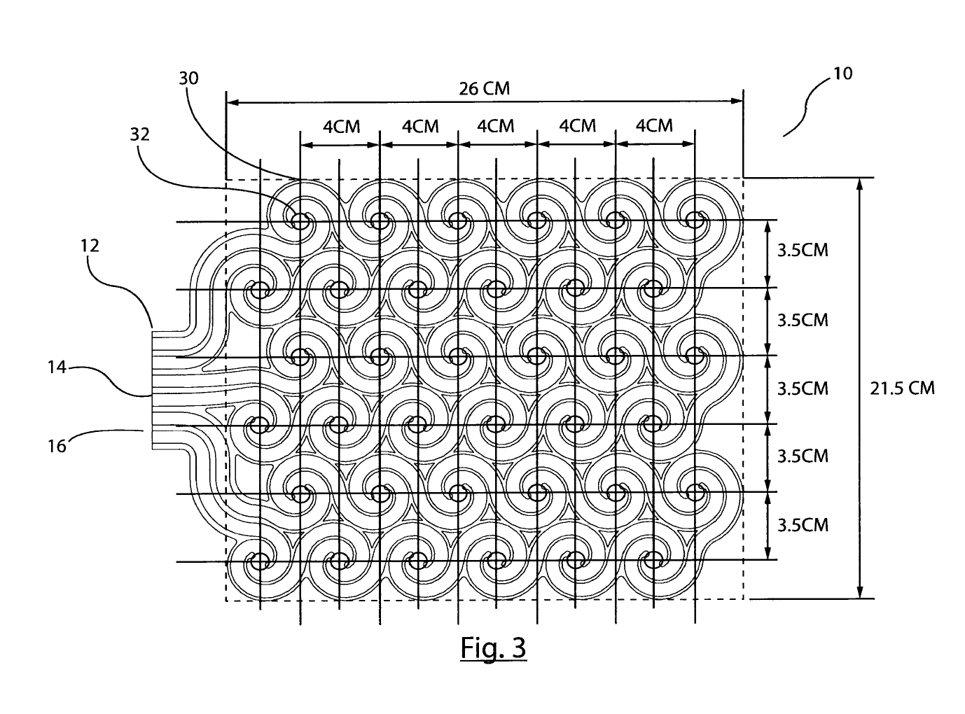

Referring now to FIG. 3, this shows in better detail the interface pack 10 of FIG. 1, some of this detail being relevant also to the embodiment of FIG. 2.

The points of path inversion 32 within the conduits 12-16 are shown by the circles in the Figure. These represent the location at which fluid within the conduits 12-16 is forced to change direction and which could be said momentarily to pause, although in practice fluid flow will continue and the pause created solely by the change in direction of the fluid. As mentioned above, the flow constrictors provided in the conduits 12-16 at the point of path inversion, specifically at the centre of each spiral 30, are pinched or otherwise narrowed in a manner similar to the embodiment of FIG. 2.

In the embodiment shown in FIG. 3, the points of fluid conversion 32 are arranged in a regular array across the surface of the interface pack 10 and thereby in practice provide a regular array of temperature regulated areas within the treatment zone of the interface pack 10. It has been found that compartmentalizing the heat transfer zones 32 in this manner provides a much more efficient structure for the transfer of heat to and/or from the interface pack 10 than prior art devices. In this embodiment, by way of illustration only, the centre points of the areas 32 are spaced around 3.5 cm to around 4 cm from one another. Specifically, the spacing between the path inversion centre points 32 is equal to the diameter of each spiral and thus around 4 cm each. On the other hand, the spacing of the relevant centre points of the spirals between the feed path and the return path of the conduits 12-16, which could be termed the Y direction, is in a region of 3.5 cm, that is around 87.5% of the spacing in the axis (X axis) orthogonal thereto.

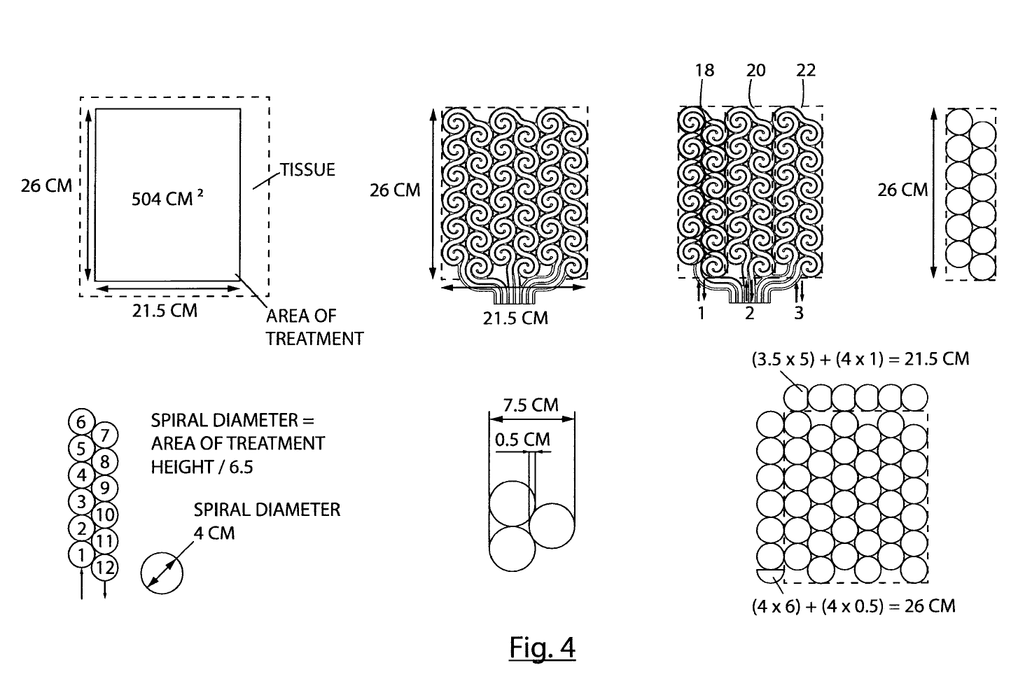

Further details of the specific embodiment shown in FIG. 3 can be found in FIG. 4. As can be seen, the interface pack 10 provides a treatment zone which is around 500 cm², with a length of around 26 cm and a width of around 21.5 cm. As explained above, the interface pack 10 is preferably made of a conformable material, such that it can be wrapped around a part of the patient's body.

As with the example of FIG. 1, the treatment zone is divided into three separate portions 18, 20, 22 with, in this example, each portion having a width in the region of 7 cm. The conduits 12-16 and spirals 30 formed in the conduits preferably provide a spiral diameter of around 4 cm.

In this embodiment, the interface pack 10 is designed to hold around 1.5 litres of fluid.

It will be appreciated that these dimensions are of a particular example only and therefore could be different for other embodiments of interface pack, for different medical applications and for the treatment of different parts of the human body.

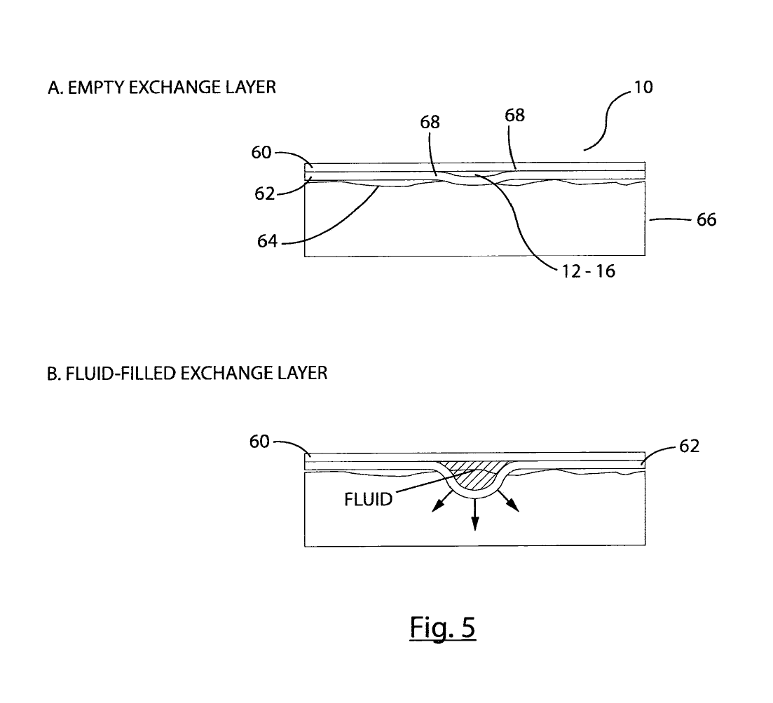

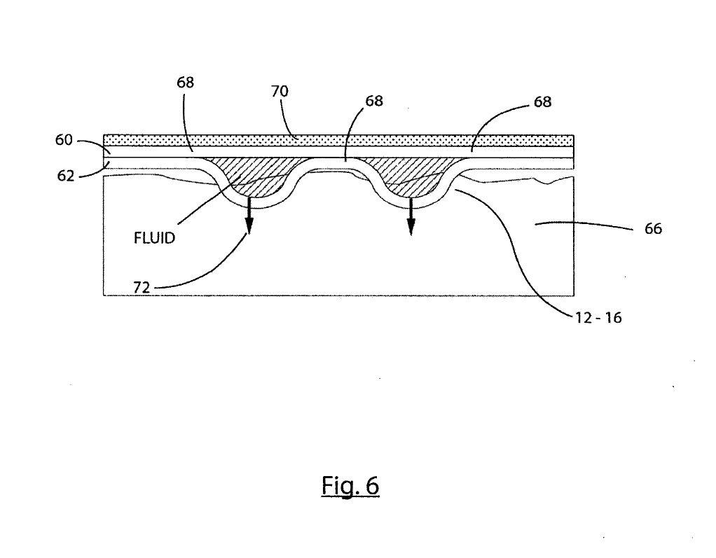

Referring now to FIGS. 5A and 5B, there is shown in schematic form a cross-sectional view of one embodiment of structure for the interface pack 10. This has of first and second layers 60, 62 of impervious material, which in this embodiment are two different materials. The first or upper layer 60 is made of a first material which is more rigid than the material of the second or contact layer 62. This contact layer 62 provides what could be described as the treatment surface 64, that is the surface of the interface pack 10 which is closest to the body 66 of the patient. The two layers 60, 62 of material are bonded to one another at bond points 68, which typically define the conduits 12-16. The bonding may be across the entire areas of the two layers 60, 62 which do not form the conduits 12-16, but may in the alternative be provided only at the edges of the conduits 12-16 so as to create these.

As will be apparent from FIG. 5B, the second or contact layer 62 is made of less rigid material so as tend to deform more than the first or upper layer 16 when fluid under pressure is fed through the conduits 12-16. In this regard, the layer 62 could be made of a material which can stretch when subjected to pressure, which would result in an increase in contact surface area during use, with the result of enhancing the heat transfer properties of the interface pack 10. This is depicted in FIG. 5B.

The layers 60, 62 could be made of different materials and also could be made of the same material, with the first layer 60 being thicker than the second or contact layer 62, such that the second layer 62 exhibits greater conformability and, in the case in which it is made of an elastic material, will stretch more than the layer 60. In one illustrative embodiment, the first layer 60 is a thermoplastic polyurethane film, for example, polyether TPU film, having a thickness of around 400 micrometres, while the second or contact layer 62, equally made of polyether TPU film, has a thickness of around 150 micrometres. Of course, any combination of different layer thicknesses and different materials may be used.

A variation of the embodiment of FIG. 5 can be seen in FIG. 6. This embodiment has first and second interface pack layers 60, 62 equivalent to the interface pack layers 60, 62 of the embodiment of FIG. 5, and has in addition a layer 70 of insulation material overlaying the interface pack layer 60. The insulation layer 70 may form the outer layer of the interface pack. The insulation layer 70 has the effect of directing energy towards the patient's tissue 66, in the direction of the arrows 72. This thus optimises energy transfer between fluid within the conduits 12-16 and the tissue 66 of the patient. It will be appreciated also that the insulation layer 70 may contribute to rigidity of the upper layer 60 of the interface pack 10, in which case it is not necessary for the layer 60 to be more rigid than the layer 62 as the additional rigidity could be provided by the insulation layer 70 alone or by a combination of the layers 70 and 60 together.

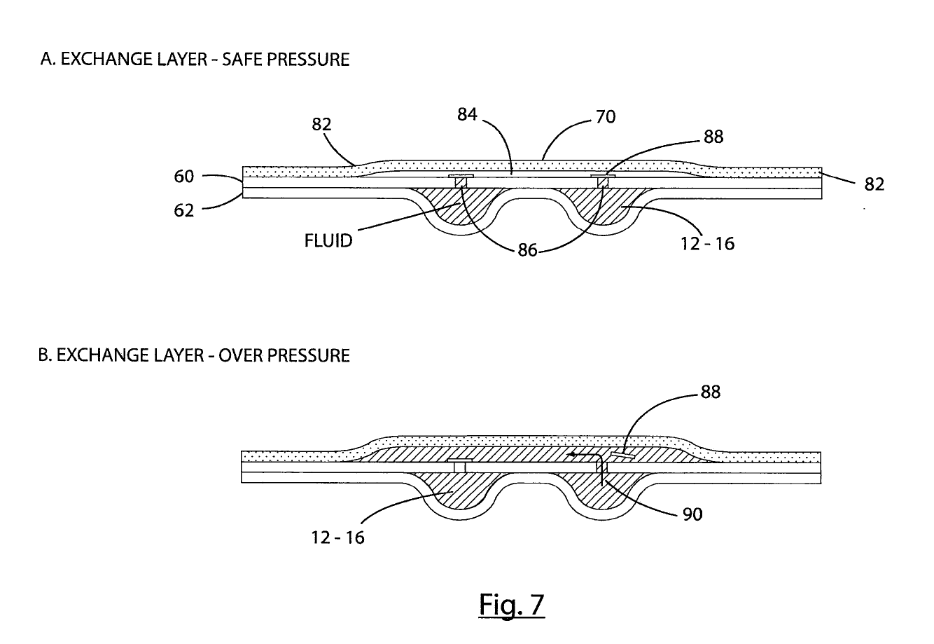

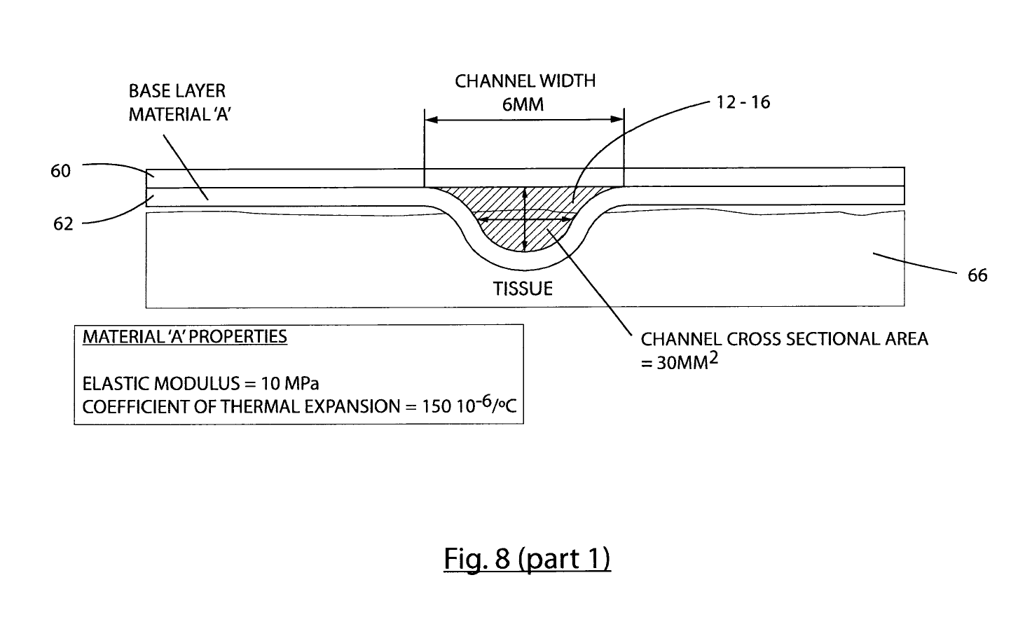

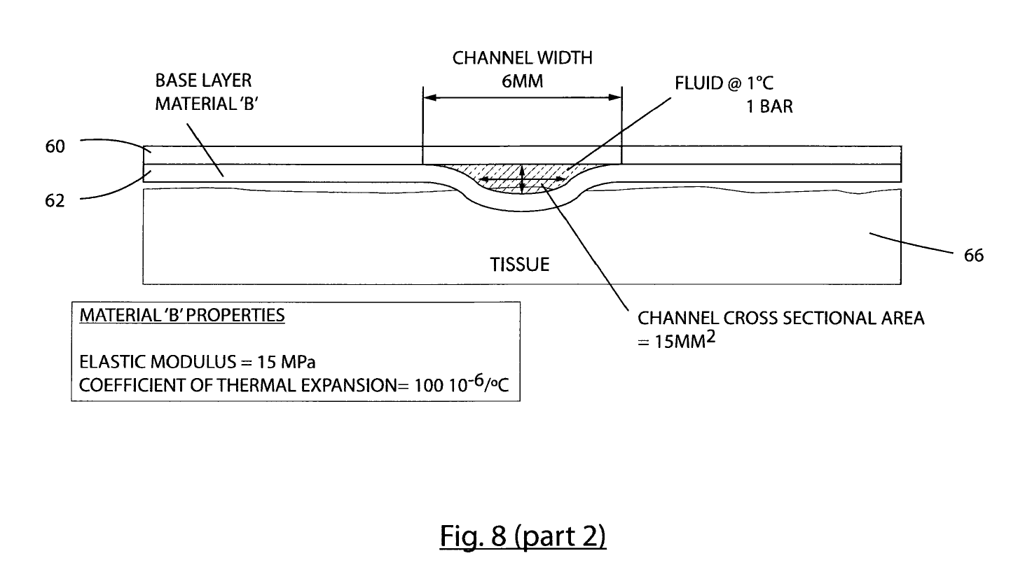

Referring now to FIGS. 7A and 7B, there is shown another embodiment of interface pack 80, which has a structure similar between embodiment of FIG. 6. Specifically, the interface pack 80 includes an insulation layer 70, a first layer 60 and a contact layer 62. The layers 60, 62 are bonded to one another so as to form the fluid conduits 12-16 as described above. The insulation 70 is bonded to the layer 60 in such a manner that it is not attached to the layer 60 in the zone overlying the regions of the conduits 12-16. This enables the creation of a space or gap 84 between the insulation layer 70 and the layer 60 of the interface pack. It will be appreciated that this gap 84 may not always be present, particularly when the interface pack 80 is pressed against a patient.

Provided in the layer 60 are a plurality of openings or holes 76 which couple the conduit 12-16 to the space between the insulation layer 70 and the interface pack layer 16. The apertures 86 are closed, in this embodiment, by adhesive patches 88, which may be small discs of material having an adhesive surface and of a size slightly larger than the size of the apertures 86. The patches 88 are designed such that they peel off the layer 60 when the pressure in the fluid in the conduits 12-16, and in particular at the apertures 86, exceeds a threshold pressure. As can be seen in FIG. 7B, when this threshold pressure is exceeded, at least one of the patches 88 will peel off to allow fluid 90 to flow out of the conduits 12-16 in order to reduce the pressure within the conduits 12-16 to a safe pressure. The released fluid is retained by the insulation layer and therefore within the structure of the interface pack, without leakage to the outside. This can ensure that the interface pack can continue to be used for that particular treatment, without it being necessary to halt the treatment prematurely in order to replace the interface pack. Such replacement can lead to an ineffective or even abortive treatment.

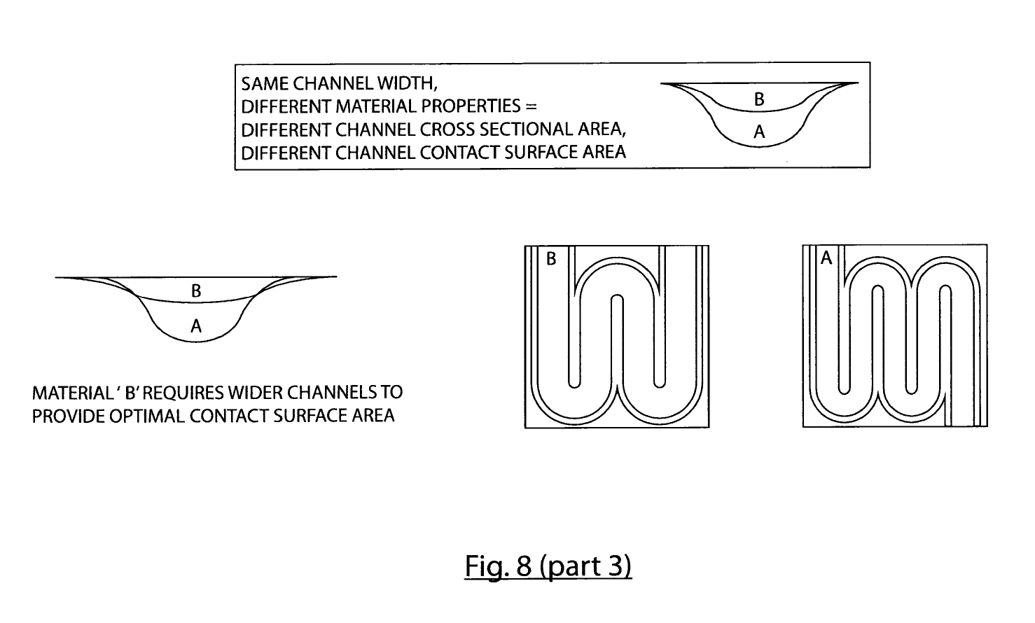

Referring now to FIG. 8, there are shown two scenarios of operation based on the use of different materials for the layers 60, 62 of the interface pack and in particular how these affect the contact area of the lower layer 62 with the patient's tissue 66.

In the upper drawing in FIG. 8, the layer 62 is made of a material “A” in which this example has elastic modulus of 10 NPa and a coefficient thermal expansion of 150×10⁻⁶/° C. In this example, when fluid at 1° C. and at a pressure of 1 bar is fed through the channels 12-16, with a channel width of 6 mm the base or lower layer 62 is able to expand so as to create a channel with a cross-sectional area of around 30 mm² which provides, as can be seen, effective and a relatively large surface area contact with the tissue 66 of the patient. By way of comparison, when the layer 62 is made of material “B”, having an elastic modulus of 15 NPa and a coefficient thermal expansion of 100×10⁻⁶/° C. As can be seen, when subjected to the same conditions the layer “B” will not stretch as much and therefore will create a channel with a cross-sectional area of only 15 mm². For the same channel width, therefore, there is a substantial reduction in the contact surface area with the patient's tissue 66. The solution to this, as can be seen in lower two sketches of FIG. 8, is to provide channels 12-16 for the lowermost example, using the material B, which are wider than the channels or conduits 12-16 of the example using material A. It is, in this regard, preferable to use for the contact or lower layer 62 a material which is more elastic and thus more conformable, in order to maximise the surface area contact with the patient's tissue. It is to be appreciated, though, that in some embodiments it may be preferable to make the layers of the interface pack of less elastic material, for example in cases where the interface pack may be subjected to particular environmental conditions not suitable to elastic material.

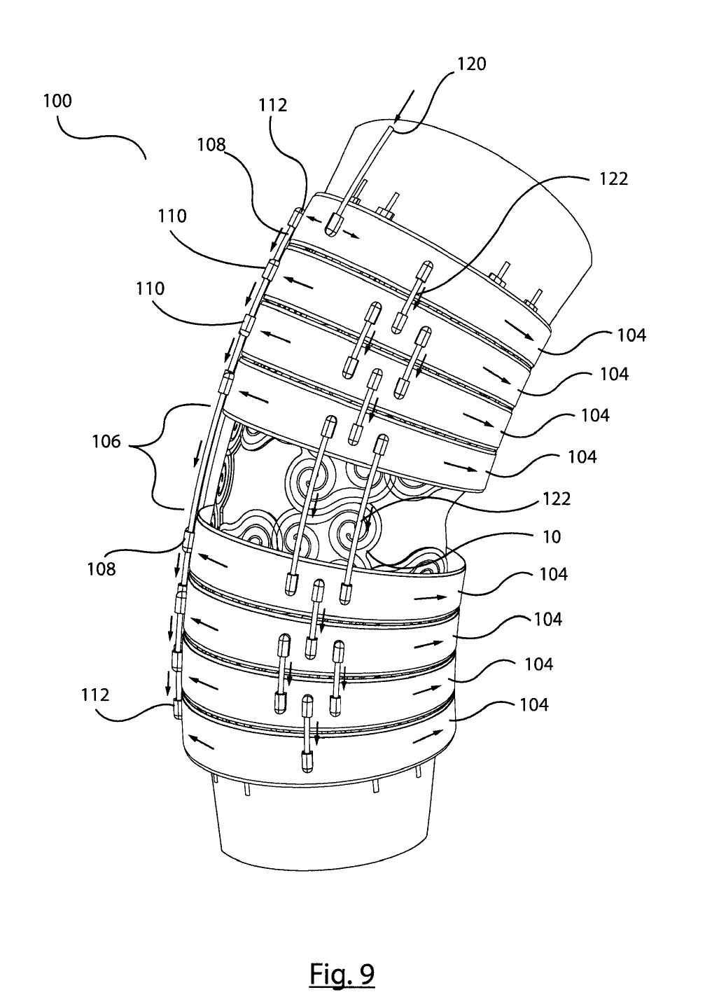

Referring now to FIG. 9, there is shown an embodiment of pressure sleeve 100 which can be used in positioning and holding of an interface pack 10 to a patient, in this example to a patient's leg 102. The pressure sleeve 100 is formed of a plurality of annular elements 104 which are in the form of annular compression straps, explained in further detail below. The pressure sleeve 100 provides, in this example, a gap 106 for accommodating a patient's knee.

The individual pressure rings 104 are coupled to one another by a rod or strut 108, which is received in guide channels 110 of the pressure rings 104, thereby to align these. The first and last pressure rings 104 in the series may be provided with closed guide channels 112 which fix to the rod strut 108, thereby to keep this in position.

In a first of the rings 104 there is provided a tube 120 for the supply of pressurised fluid, typically air, into the compression rings 104. As can be seen in FIG. 9, there are also provided coupling tubes 122 from one compression ring 104 to the other. There may be provided a plurality of coupling tubes 122 between adjacent coupling rings, as shown and also, as necessary, circumferentially spaced around the rings 104.

Referring now to FIG. 10, a part of the pressure sleeve 100 of FIG. 9 is shown in the two cross-sectional views of FIG. 10. The upper view of FIG. 10 shows two of the annular pressure rings 104 in what could be termed a non-compressive state. The rings 104 are formed of a substantially rigid outer layer 126, typically made of a substantially rigid plastics material. Disposed annually around the inside of layer 126 is a sleeve 128 made of a soft conformable material and preferably of an elastic material such as polyether TPU film having a thickness of around 80 micrometres. The sleeve 128 preferably extends for the entire circumference of the outer layer 126. The layer 128 can be considered an annular pouch or ring having an annular cavity. The tubes 120, 122 are coupled to the cavity of the compression layer 128. Thus, as can be seen in the lowermost sketch of FIG. 10, when air is supplied through tube 120 under pressure, this causes the cavity of the layer 128 to expand, as can be seen from arrows 130, in practice expanding against the patient 102. Fluid from the upper layer 128 passes via the tubes 122 into the equivalent layer or chamber 128 of the second compression ring 104 in the series (and, as will be appreciated, all of the other subsequent compression rings 124 of the pressure sleeve 100).

It will be appreciated that the compression of the layers or chambers 128 will apply pressure against the patient's body, thereby ensuring that the interface pack 10 is firmly held in position. Furthermore, the pressure sleeve 100 can apply therapeutic compressive pressure to the patient's body, useful in treating many ailments.

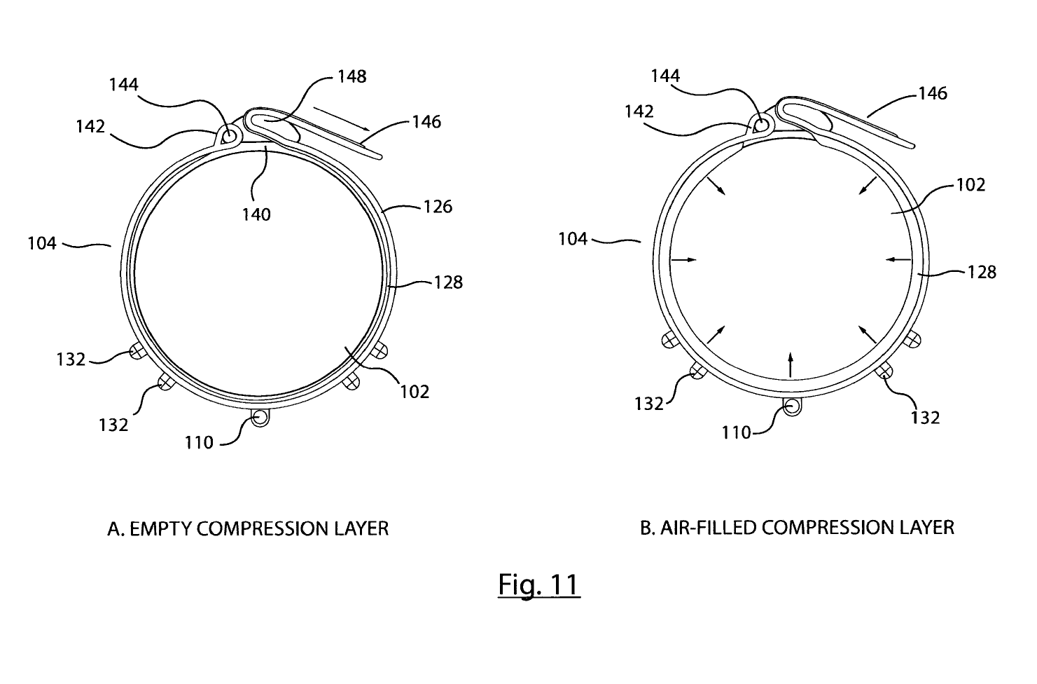

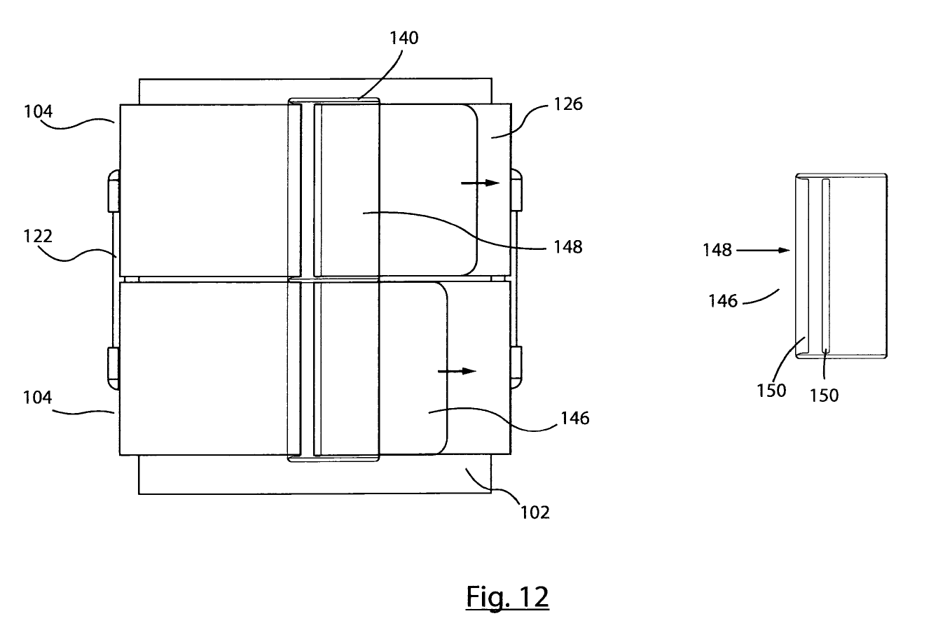

Referring now to FIG. 11, this shows a plan view of one of the pressure rings 104. The pressure ring 104 has the structure shown in FIGS. 9 and 10, namely with an outer substantially rigid layer 126, a compression layer or chamber 128, guide channels 110 for receiving the support rod or strut 108, and fluid coupling ports 132 for receiving the feed tubes 122. In addition, FIG. 11 shows the provision of an adjustment clip 140 for use in adjusting the length of the pressure rings 140 to suit the dimensions of the patient, in this example the patient's leg 102. The clip 140 can also be seen in FIG. 12. The pressure rings 104 are in the form of straps, having one end 142 looped and held within a support rod 144 of the clip 140. A second end 146 can loop around a latch element 148 of the clip 140, in particular by being inverted around the latch element 148 and in the preferred embodiment compressed flat by being fed into respective slots 150 of the clip 148. Clips or buckles suitable for the clip 140 will be apparent to the person skilled in the art.

In practice, the clip 148 not only holds the second end 146 of the compression ring 140 tied in position but it also compresses it flat to close off the compression chamber formed by the layer 128. Thus, as can be seen in the lower drawing of FIG. 11, when fluid under pressure is fed into chamber 128, this can expand relatively inwardly in the direction of the arrows shown in FIG. 11 but there is no expansion of any portion of the chamber 128 beyond the position of the clip 140, by virtue of this being pressed closed by the clip.

This arrangement allows for the provision of an adjustable compression sleeve in which only that portion of the sleeve which lies against the patient is expanded under pressure, with any excess parts of that sleeve being closed off from the compression fluid.

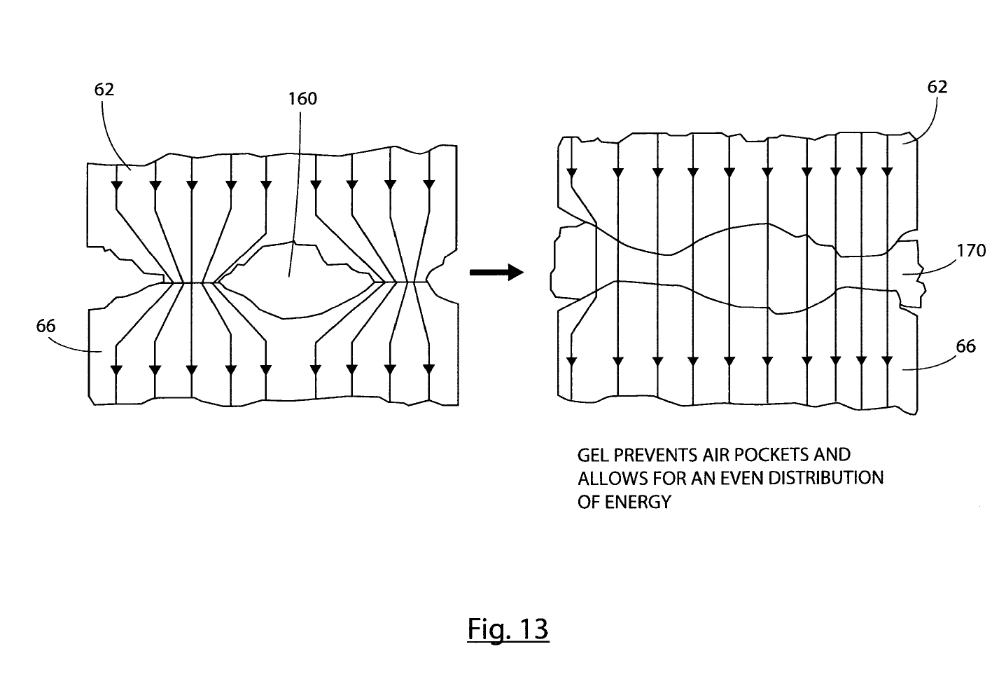

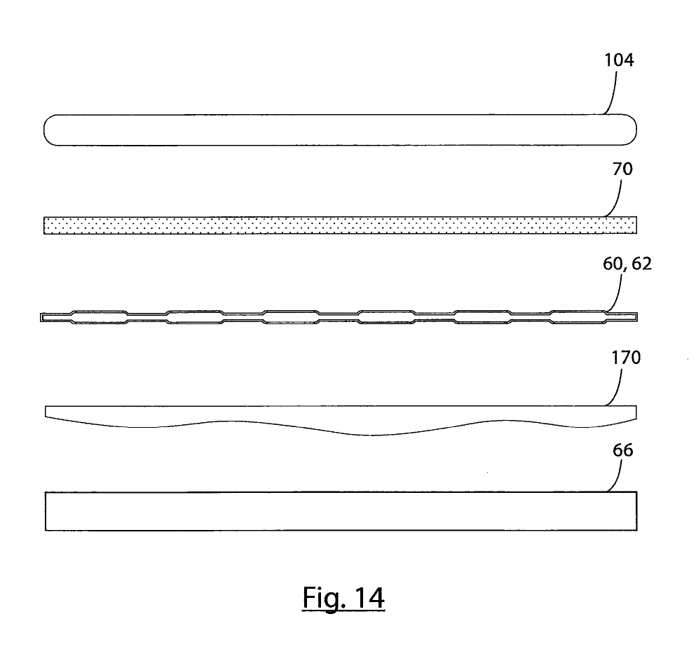

Referring now to FIG. 13, there is shown a further enhancement to the thermoregulation interface pack structure taught herein. The left hand drawing of FIG. 13, which is of schematic form, shows that under some circumstances there may be air gaps 116 between the contact layer 62 of the interface pack 10 and the patient's skin 66, caused by irregularities in the patient's skin surface and in a non-conforming shape of the interface pack 10. In this embodiment, a layer of thermally conductive gel 170 is disposed between the patient's skin 66 and the contact layer 62 of the interface pack. This, as can be seen in the right hand sketch of FIG. 13, will fill in any air gaps between the interface pack and the patient's skin, thereby ensuring good and continuous contact between the patient and the interface pack. The resultant structure can be seen in schematic form in FIG. 14, which includes the compression sleeve 104, the layer of insulation 70, the interface pack layers 60, 62 and the gel layer 170 all pressed against the patient's skin 66.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosure in the abstract accompanying this application is incorporated herein by reference.

1-22. (canceled)

23. A thermoregulation interface pack provided with:
a. a treatment surface having a treatment zone;
b. at least one fluid conduit in the interface pack and extending across the treatment zone, each fluid conduit including:
(1) path inversions disposed in the treatment zone, and
(2) flow constrictors disposed proximate at least some of the path inversions, wherein each of the path inversions provides a zone of zero apparent fluid flow when fluid is flowing in the fluid conduit.

24. The thermoregulation interface pack of claim 23 wherein flow constrictors are provided at or adjacent each path inversion in the treatment zone.

25. The thermoregulation interface pack of claim 24 wherein the flow constrictors are located at the path inversions.

26. The thermoregulation interface pack of claim 23 wherein the path inversions are uniformly disposed in a regular array about the treatment zone.

27. The thermoregulation interface pack of claim 23 wherein the treatment zone extends for the entirety of the treatment surface of the interface pack.

28. The thermoregulation interface pack of claim 23 wherein each conduit is in the form of a series of spirals, with spirals on opposite sides of each path inversion curving in opposite directions.

29. The thermoregulation interface pack of claim 23 wherein the flow constrictors are in the form of a narrowing of the conduit.

30. The thermoregulation interface pack of claim 23 wherein the interface pack includes first and second layers forming the interface pack, the second layer defining the treatment surface and the first layer defining an outer layer of the interface pack.

31. The thermoregulation interface pack of claim 30 wherein the first layer has a stiffness greater than a stiffness of the second layer at least in the treatment zone.

32. The thermoregulation interface pack of claim 30 wherein the second layer is:
a. made of a conformable material, and/or
b. thinner than the first layer.

33. The thermoregulation interface pack of claim 30 wherein the first and second layers are formed of different materials.

34. The thermoregulation interface pack of claim 23 further including an insulation layer.

35. The thermoregulation interface pack of claim 34 wherein the insulation layer is disposed across an outer layer of the interface pack.

36. The thermoregulation interface pack of claim 23 wherein the interface pack is provided with one or more pressure relief valves.

37. The thermoregulation interface pack of claim 36 wherein two or more pressure relief valves are provided within each conduit.

38. The thermoregulation interface pack of claim 36 wherein the pressure relief valves are covered by an insulation layer.

39. The thermoregulation interface pack of claim 23 further including a compression element for pressing the treatment surface against a patient.

40. The thermoregulation interface pack of claim 39 wherein the compression element is a pressure sleeve or belt.

41. The thermoregulation interface pack of claim 39 wherein the compression element includes several compression belts arrayed lengthwise along the interface pack.

42. The thermoregulation interface pack of claim 23 wherein a gel layer is disposed over the treatment surface for contact with a patient.

43. A thermoregulation interface pack having a fluid conduit extending along a treatment surface, the fluid conduit including:
a. a forward path,
b. a path inversion following the forward path,
c. a return path following the path inversion, wherein fluid flowing within the fluid conduit in a first direction along the forward path then flows in an opposite direction along the return path,
d. a flow constrictor situated at or adjacent to the path inversion, wherein the flow constrictor is configured to generate turbulence in fluid flowing within the fluid conduit at the path inversion.

44. A thermoregulation interface pack having a fluid conduit extending along a treatment surface, the fluid conduit including:
a. a forward path wherein fluid flows along a forward direction,
b. a return path wherein the fluid flows along a return direction oriented at least substantially opposite the forward direction,
c. a path inversion situated between the forward and return paths,
d. a flow constrictor partially obstructing flow of the fluid within the fluid conduit at or adjacent to the path inversion.
